(19) United States
(12) Patent Application Publication
(10) Pub. No.: US 2006/0069376 A1
(43) Pub. Date: Mar. 30, 2006

(54) Absorbent article with extended side panels

(76) Inventors: Elizabeth Miller, King of Prussia, PA (US); Dane Jackson, Gilbertsville, PA (US); Nicole Divittore, West Chester, PA (US)

Correspondence Address: RATNERPRESTIA, P O BOX 980, VALLEY FORGE, PA, 19482-0980, US

(21) Appl. No.: 10/955,672

(22) Filed: Sep. 30, 2004

(51) Int. Cl. A61F 13/15

(52) U.S. Cl. 604/385.201; 604/400

(57) ABSTRACT

The invention includes an absorbent article comprising a front section and a back section. The front and back sections are each configured to be positioned against the front and back, respectively, of a wearer of the absorbent article. At least one extension extends between the front and back sections, and the extension has ends coupled to the front and back sections. The front and back sections and the extension together define a continuous waist of the absorbent article. A fastener is applied to the article, and a portion of the waist folds together to adjust the size of the waist.

### BACKGROUND OF THE INVENTION

Absorbent articles which effectively contain body exudates and are capable of being pulled up or down over the hips of the wearer are desirable for specific applications, such as to assist in toilet training children. However, absorbent articles such as training pants have not always been able to achieve a close conforming fit to the wearer while still being able to expand enough to be pulled up and down over the hips of the wearer. For example, if training pants fit the waist of a wearer too loosely, loose fitting pants can undesirably result in leaks. As a result, such training pant articles would not contain bodily exudates as effectively as conventional diaper-type articles, which can be adjusted to achieve a more conforming fit to the wearer. Moreover, the removal of soiled conventional training pants has not always been completely satisfactory as they often do not provide adequate room for convenient removal and can be cumbersome to dissemble.

### SUMMARY OF THE INVENTION

The invention includes an absorbent article comprising a front section and a back section. The front and back sections are each configured to be positioned against the front and back, respectively, of a wearer of the absorbent article. At least one extension extends between the front and back sections, and the extension has ends coupled to the front and back sections. The front and back sections and the extension together define a continuous waist of the absorbent article. A fastener is applied to the article, and a portion of the waist folds together to adjust the size of the waist. The fastener is releasably attachable to the waist, thereby securing the adjusted size of the waist.

The invention further includes a method of making an absorbent article comprising forming a center portion of the absorbent article. The center portion has a front section and a back section, and the front and back sections are each configured to be positioned against the front and back, respectively, of a wearer of the absorbent article. The method includes coupling the ends of an extension to the front and back sections, creating a continuous waist of the absorbent article comprising the front and back sections and the extension. The extension allows a portion of the waist to fold together to adjust the size of the waist.

### BRIEF DESCRIPTION OF THE DRAWINGS

Exemplary embodiments of this invention will be described with reference to the drawing, of which:

FIG. 1 is a top view of an absorbent article according to an exemplary embodiment of the present invention.

FIG. 2 is a top view of the absorbent article of FIG. 1 illustrating a continuous waist according to an exemplary embodiment of the present invention illustrating.

FIG. 3 is a top view of an absorbent article according to another exemplary embodiment of the present invention.

FIG. 4A is a top view of an absorbent article according to yet another exemplary embodiment of the present invention.

FIG. 4B is a cross-sectional view of the absorbent article of FIG. 4A.

FIG. 5A is a top perspective view of an absorbent article according to yet another exemplary embodiment of the present invention.

FIG. 5B is a cross-sectional view of the absorbent article of FIG. 5A.

FIG. 6 is a cross-sectional top view schematic of the absorbent article of FIG. 4A.

FIG. 7 is a cross-sectional top view schematic of the absorbent article of FIG. 5A.

FIG. 8 is a cross-sectional top view schematic of an absorbent article according to still another exemplary embodiment of the present invention.

FIG. 9A is a top view schematic of an absorbent article according to another exemplary embodiment of the present invention.

FIG. 9B is a top view schematic of the absorbent article of FIG. 9A.

FIG. 9C is a cross-sectional top view schematic of the absorbent article of FIG. 9A in the unfastened position.

FIG. 9D is a cross-sectional top view schematic of the absorbent article of FIG. 9A in the fastened position.

FIG. 10A is a perspective view of an absorbent article in the fastened position according to an exemplary embodiment of the present invention.

FIG. 10B is a perspective view of the absorbent article of FIG. 10A in the unfastened position.

FIG. 11A is a perspective view of an absorbent article in the fastened position according to another exemplary embodiment of the present invention.

FIG. 11B is a perspective view of the absorbent article of FIG. 11A in the unfastened position.

FIG. 12 is a schematic illustration of a side extension of an absorbent article according to another exemplary embodiment of the present invention.

### DETAILED DESCRIPTION OF THE INVENTION

The invention is best understood from the following detailed description when read in connection with the accompanying drawing, which shows exemplary embodiments of the invention selected for illustrative purposes. It is emphasized that, according to common practice, the various features of the drawing are not to scale. On the contrary, the dimensions of the various features may be arbitrarily expanded or reduced for clarity.

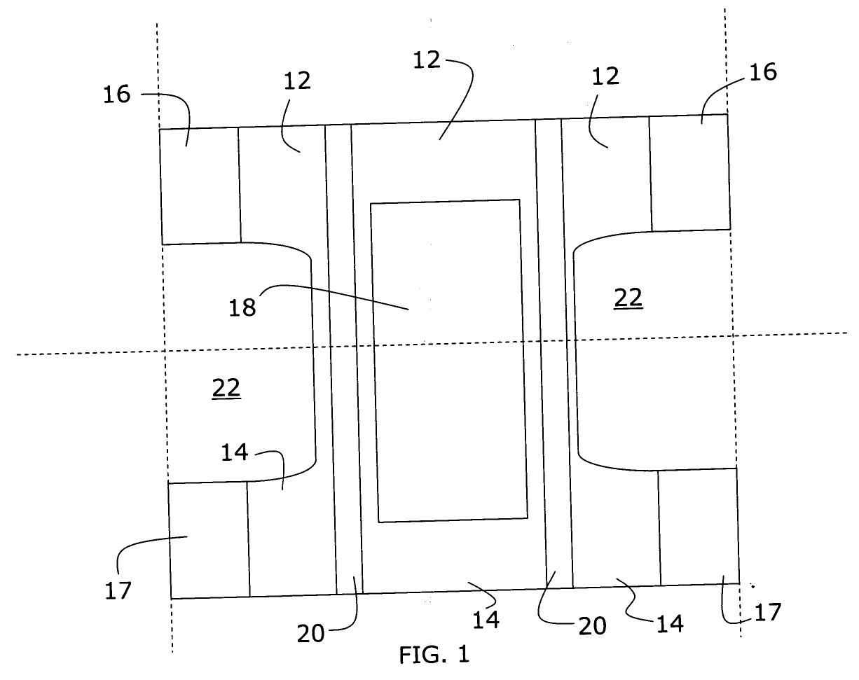

Referring generally to the drawing (FIGS. 1-12), an absorbent article comprises a front section 12 and a back section 14, and the front and back sections are each configured to be positioned against the front and back, respectively, of a wearer of the absorbent article. The absorbent article includes at least one extension 15 extending between the front and back sections. The extension has ends coupled to the front and back sections, and the front and back sections and the extension together define a continuous waist of the absorbent article. A fastener 36 is applied to the absorbent article, and a portion of the article's waist folds together to adjust the size of the waist. The fastener is releasably attachable to the waist, thus securing the adjusted size of the waist. The fastener may be applied to a flap 38, which is a continuation of the back section of the absorbent article.

The absorbent article is formed by forming the center portion, which has a front section 12 and a back section 14. The front and back sections are each configured to be positioned against the front and back, respectively, of a wearer of the absorbent article. The ends of an extension 15, which extends between the front and back sections, are coupled to the front and back sections. This creates a continuous waist of the absorbent article from the front and back sections and the extension. The extensions allow a portion of the waist to fold together to adjust the size of the waist. A fastener 36 is attached to the article in a location such that the fastener functions to secure the waist in the adjusted size. In another embodiment, a flap 38 is formed from the back section 14 by attaching one end of the extension 15 to the back section 14 in a bond region 27 of the back section 14. The flap is formed from a portion of the back section between the bond region and the end of the back section.

Referring now to the drawing in particular, in which like reference numbers refer to like elements throughout, FIG. 1 shows a top view of an absorbent article according to one exemplary embodiment of the present invention. The absorbent article of FIG. 1 is shown disconnected at the waist. A center portion of the article has a front section 12 and a back section 14, which are each configured to be positioned against the front and back, respectively, of a wearer of the absorbent article. The front and back section make up an absorbent chassis, and include an absorbent core 18, contained in an interior and exterior cover sheet. The interior and exterior cover sheets are made of materials appropriate for use in absorbent articles generally.

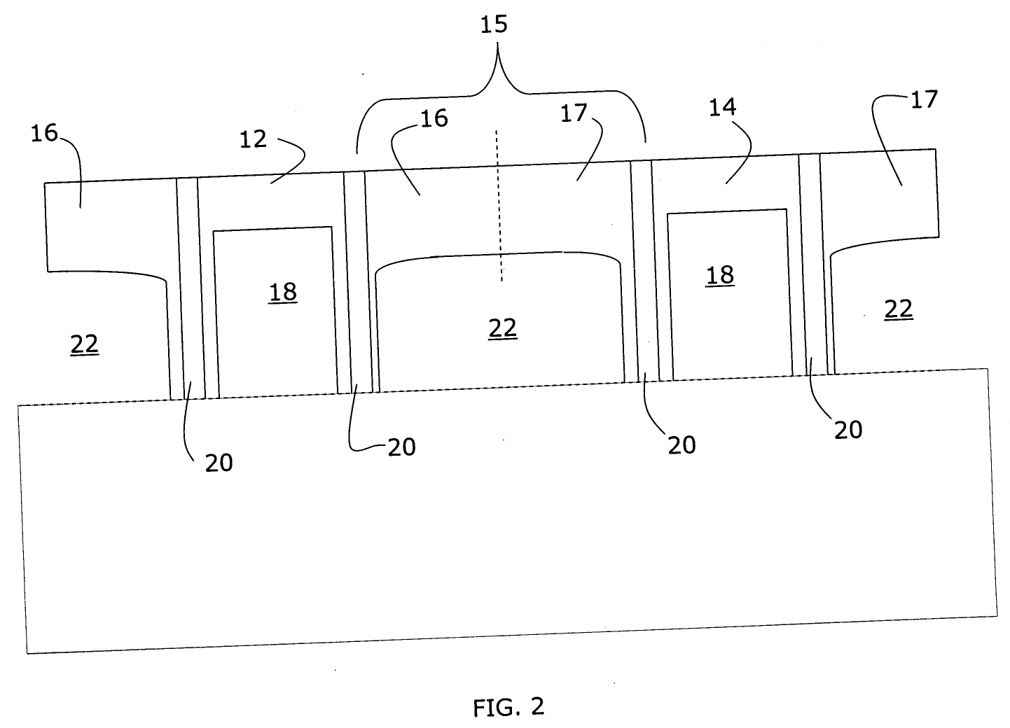

As shown in FIG. 1, the center portion contains leg gathers 20 between an absorbent core 18 and the perimeter of the article defining leg holes 22. Leg gathers may be formed by elasticizing a designated portion of the front and back sections. The vertical dotted lines of FIG. 1 signify the location of a cut in extensions between the front section 12 and the back section 14. The front portion of the extension 16 is shown attached to the front section 12 and the back portion of the extension 17 is shown attached to the back section 14. The front and back portions of the extension together make up the extension 15, as shown in FIG. 2.

FIG. 2 shows a top view of the absorbent article of FIG. 1 with a connected waist on one side. The front section 12 and the back section 14 are connected by the extension 15. The extension 15 is shown encompassing the front portion of the extension 16 and the back portion of the extension 17 with the portions differentiated by a short dashed line. The extension 15 may be formed of a single continuous piece of material, or a combination of two pieces of material bonded together to form a continuous extension between the front section 12 and the back section 14.

The extension may be formed from elastic or non-elastic material. For example, the extension may include various woven or nonwoven materials such as spunbond material, meltblown material, cotton material, rayon material or combinations thereof such as a spunbond-meltblown-spunbond (SMS) laminate material. In one exemplary embodiment of the invention, the extension is a breathable, non-woven material.

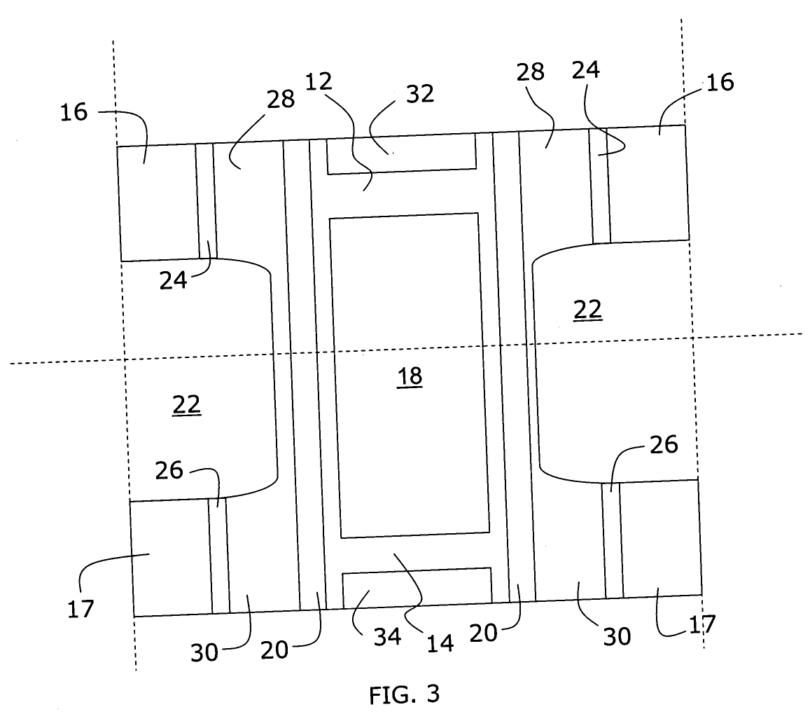

FIG. 3 illustrates an additional exemplary embodiment of the absorbent article. A bond 24 between the front portion of the extension 16 and a front stretch panel 28 is shown, as well as a bond 26 between the back portion of the extension 17 and a back stretch panel 30. A bond may be formed between two or more materials through adhesive, thermal, thermal-mechanical or ultrasonic bonding techniques known to those skilled in the art.

The front and back stretch panels may be formed of any elasticized material, including a stretch-thermal laminate (STL), neck-bonded laminate (NBL), or stretch-bonded laminate (SBL) material. While the front stretch panel 28 and the back stretch panel 30 may be formed of a separate piece of material than the front section 12 and the back section 14 respectively, and bonded to their respective sections, they are shown in FIG. 3 as being formed of the same material as the front and back sections. Accordingly, the front and back side panels may be part of a unitary core or unitary absorbent article. Throughout this description, unless specifically noted otherwise, the stretch panels are presented as a part of the front or back sections. In one exemplary embodiment, the front stretch panel 28 and the back stretch panel 30 are elasticized regions of the front section 12 and the back section 14 respectively.

Again referring to FIG. 3, a front waistband 32 and a back waistband 34 are shown as regions of the front and back sections respectively. These regions may include additional elastic material and/or structural support materials.

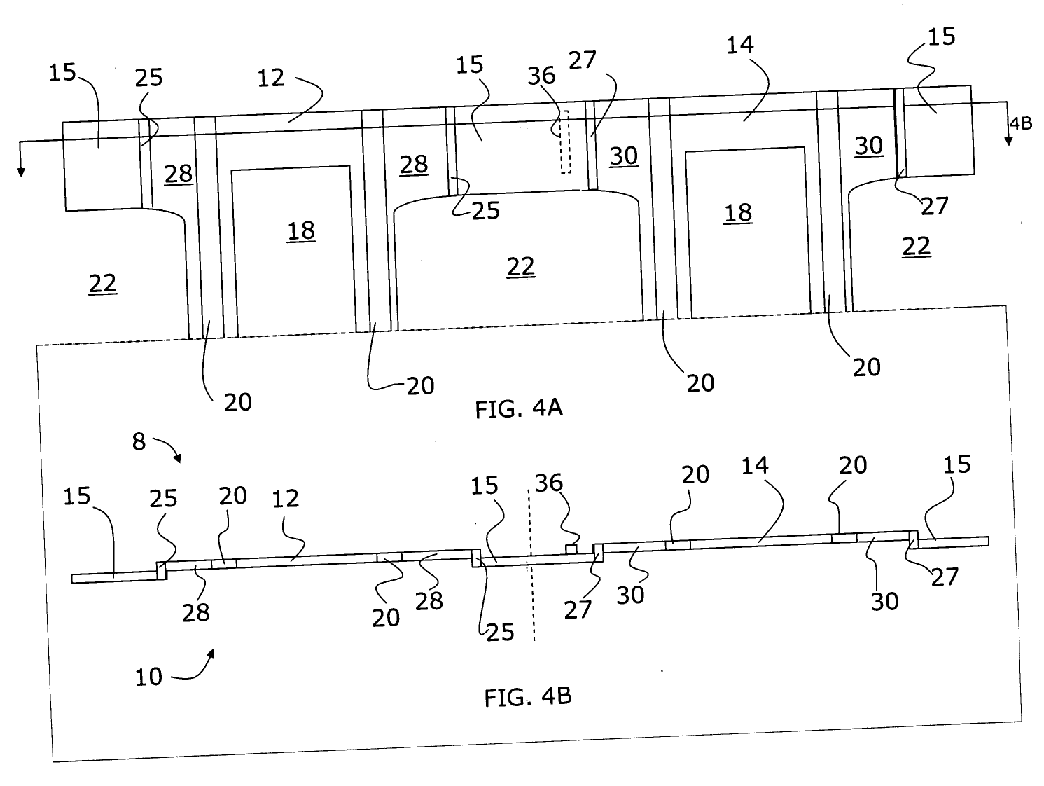

Now referring to FIG. 4A, the absorbent article of FIG. 3 is shown with an attached waist on one side of the wearer, and with a cut along the dotted horizontal line of FIG. 3. An extension 15 is shown bonded to the front stretch panel 28 through a bond 25 and bonded to the back stretch panel 30 through a bond 27. Shown in dashed lines indicating its position on the outboard side of the absorbent article is a fastener 36 attached to the exterior side of the extension.

FIG. 4B is a cross-sectional view of article in FIG. 4A. In this exemplary embodiment, the front section 12, the leg gathers 20, and the front stretch panel 28 are formed from the same material, without any bonds. The extension 15 is connected at its ends to the front 28 and back 30 stretch panels through bonds 25, 27 respectively. The fastener 36 is shown attached to the exterior side 8 of the absorbent article. The exterior side of the article faces away from the wearer when worn, whereas the interior side 10 is against the wearer when in use.

The extension 15 can vary in length depending on the size of the absorbent article. The use of the extension is appropriate in absorbent articles designed for small children, as well as those designed for adult users. The length of the extension will vary on the sizing of the absorbent article. In one exemplary embodiment, the extension has a length sufficient to permit facile positioning and removal of the absorbent article in an unfastened configuration. Generally, the extension 15 allows for the waist of the article to expand sufficiently so that the article may be positioned on the wearer and removed from the wearer without disrupting the continuity of the waist. The extension folds into the interior side of the waist of the article when the waist of the article is adjusted to the wearer, and secured in the fastened configuration.

The extension may include a single component or multiple components, such as a non-stretchable component and a stretchable component. Non-stretchable components provide adjustability by folding together when the waist is adjusted to fit the wearer. In one exemplary embodiment, stretchable extensions extend between the front and back sections on both sides of the article.

The extensions may include various woven or non-woven materials such as spunbond material, such as a spun-bonded polypropylene or polyester material, or a bi-component spun-bonded material such as a spun-bonded polyethylene and polypropylene material; meltblown material; cotton material; rayon material; or combinations thereof such as a spunbond-meltblown-spunbond (SMS) or spunbond-spunbond (SS) laminate material. Additionally, the extension could be formed from a film or fabric material.

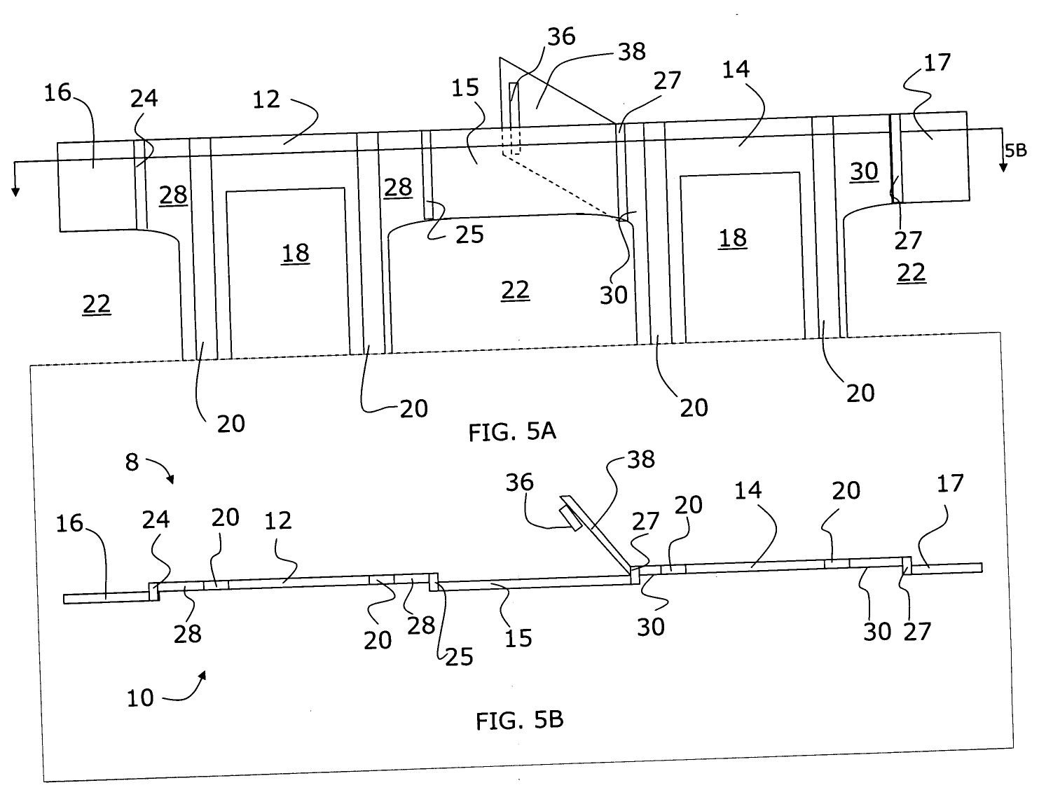

Another exemplary embodiment is shown in FIG. 5A in which a flap 38 is formed from a portion of the back section 14, shown in FIG. 5A as a portion of the back stretch panel 30. The flap 38 is formed when the extension 15 is attached at its end through a bond 27 to the back section. The remaining portion of the back section between the bond location and the end of the back section generates the flap material. In one exemplary embodiment, the flap 38 has a fastener 36 attached to the flap such that the fastener can fasten the flap to the exterior side of the article.

FIG. 5B is a cross-sectional view of the absorbent article in FIG. 5A.

While the flap 38 is formed by the bond 27 between the extension 15 and the back stretch portion 30, the flap may be a continuous piece of material with the back stretch portion 30 and the back section 14, as shown in FIG. 5B, or the flap may alternatively be a separate piece of material bonded to the article. The flap contains a fastener facing the article.

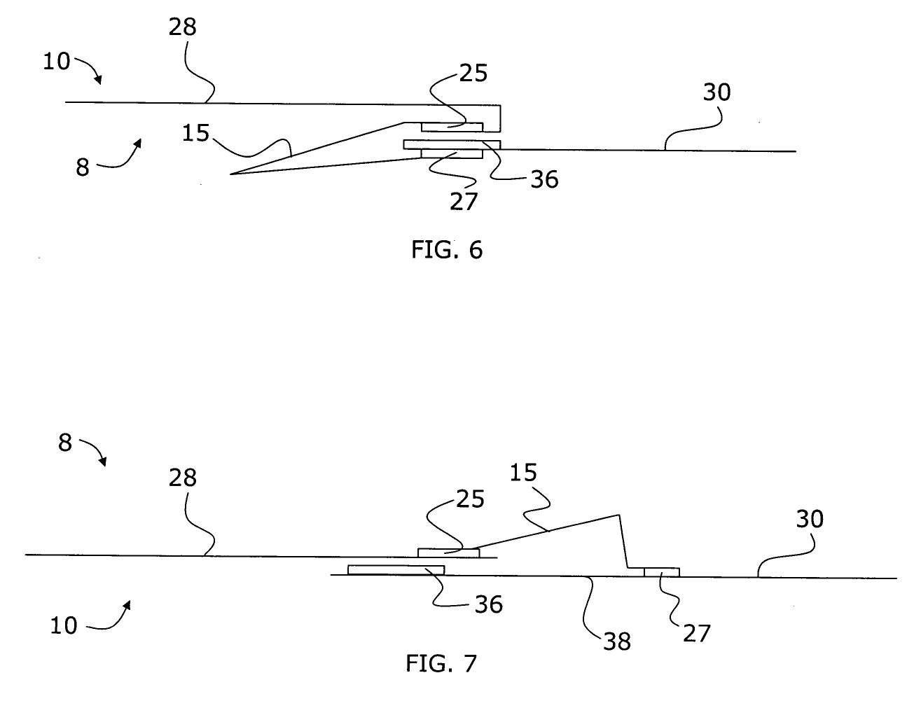

The invention provides for several fastener configurations consistent with serving the function of securing the waist of the article in the position adjusted to the wearer. A fastener configuration for the absorbent article of FIG. 4A is shown schematically in FIG. 6. The extension 15 folds to provide adjustability in the circumference of the waist, and the front section's stretch panel 28 is fastened to the fastener 36 on the back section's stretch panel 30. The fastener could alternatively be located on the front section and fasten to the extension or back section. The fastener may be a mechanical fastener, such as a hook and loop fastener system, or an adhesive fastener.

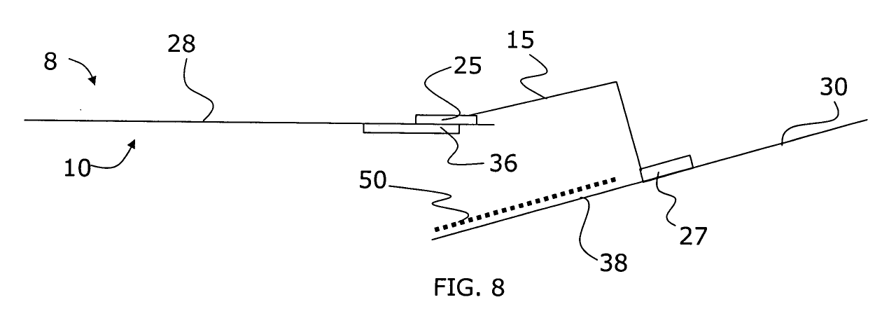

FIG. 7 is a schematic top view of the embodiment shown in FIG. 5A. The article is shown with the extension in an intermediary state between a fully extended unfastened position and a fastened position, as shown in FIG. 6. As shown in FIG. 7, the fastener 36 is attached to a flap 38 formed from the back stretch panel 30 material. The extension folds to provide adjustability in waist circumference, and the fastener 36 on the flap 38 fastens to a location on the extension 15 or the front stretch panel 28, depending on the amount of folding required for proper adjustment.

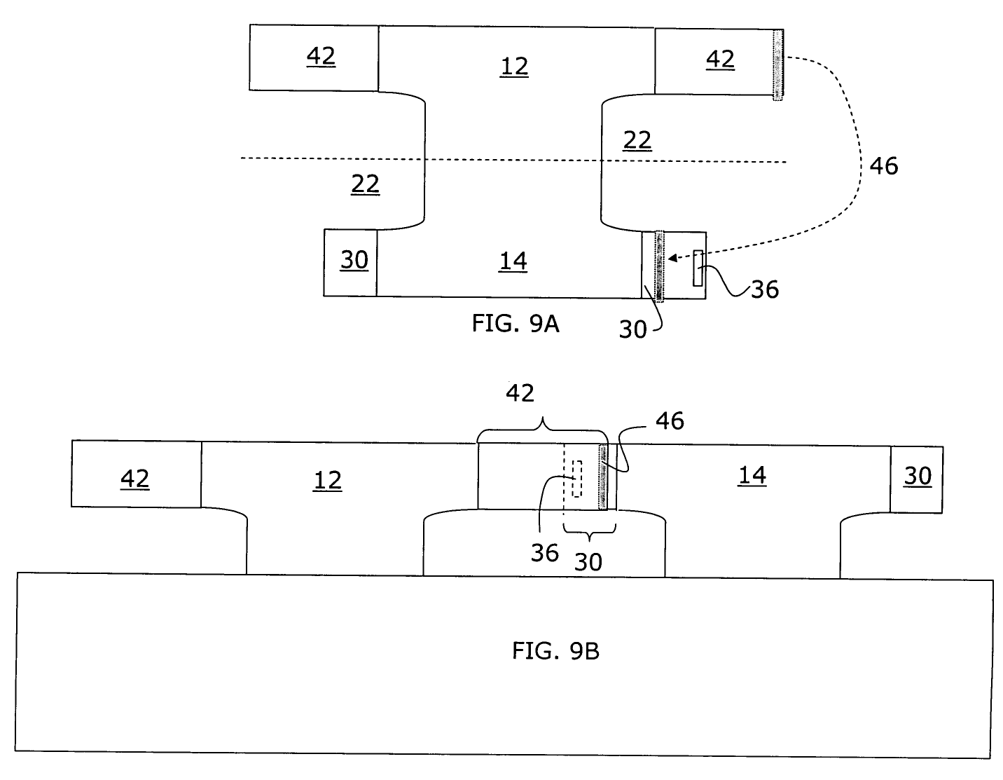

Referring to FIG. 8, which shows another embodiment, the fastener 36 fastens to the fastener's complementary component 50, such as a landing zone. The fastener 36 is shown attached to the exterior side 10 of the front stretch panel 28 in FIG. 8 with the complementary fastener component 50 positioned on the flap 38 formed from the back stretch panel 30. The fastener can also be positioned on the flap, with its the complementary component on the extension, front stretch panel, or front section.

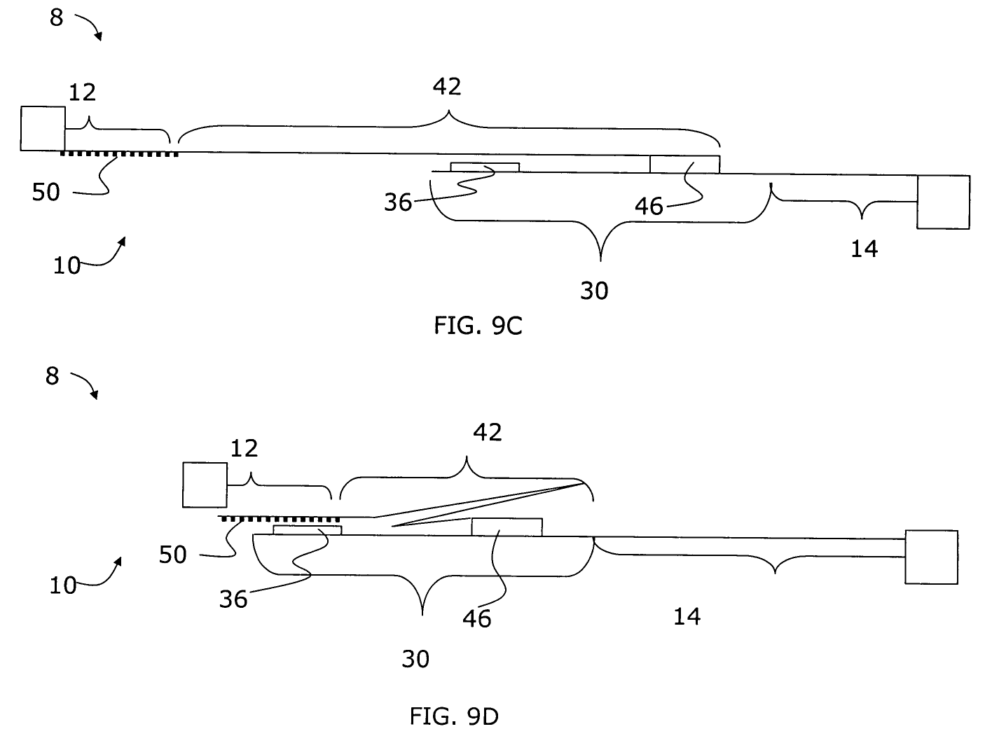

FIG. 9A is a top view of another exemplary embodiment of the absorbent article of the invention in which the front section 12 extends into an extended front stretch panel 42. The extended front stretch panel extends from the front section 12 further than the front stretch panel 28, and further than the back stretch panel 30 extends from the back section 14 to form an asymmetric article in this view, with the line of asymmetry shown as a dashed line in FIG. 9A. In this embodiment, the extended front stretch panel 42 serves as the extension between the front and back sections, that is adjustable with a fastener. As shown in FIG. 9A, the fastener 36 is attached to the back stretch panel 30. The end of the back stretch panel 30 forms a flap when the extended front stretch panel 42 is bonded to the back stretch panel 30 through a bond 46. FIG. 9B shows the continuous waist formed when the extended front stretch panel 42 is attached to the back stretch panel 30 through a bond 46.

Another view of the continuous waist in the unfastened configuration is shown in FIG. 9C, with the fastened configuration shown in FIG. 9D. The extended front stretch panel 42 is able to fold up upon itself and the other components of the continuous waist to provide adjustability. The fastener 36 fastens the back stretch panel to the fastener's complimentary component 50, which is shown positioned on the front section 12, but may be positioned on the extend front stretch panel 42 as well.

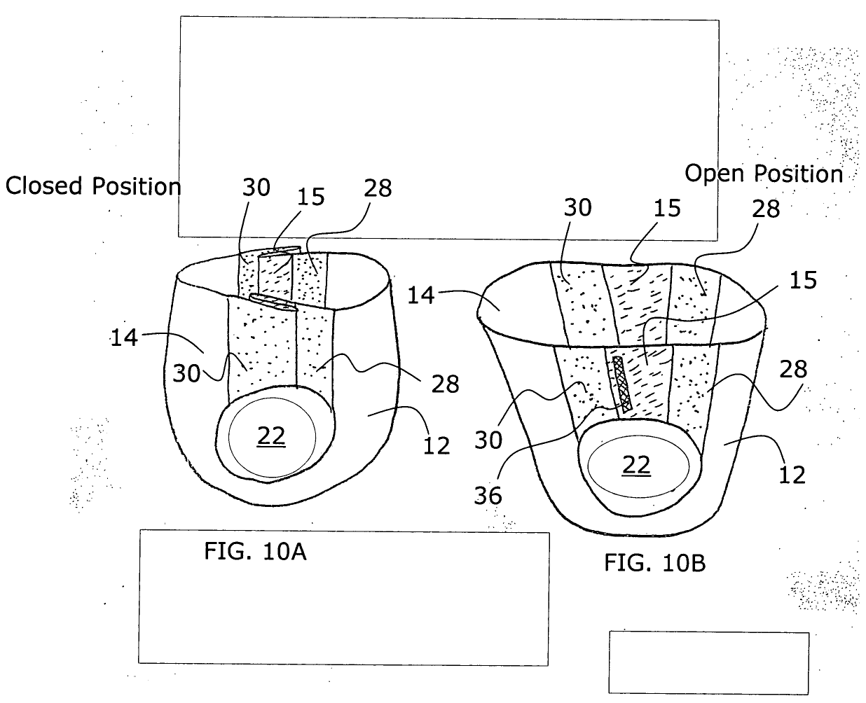

FIGS. 10A and 10B show an exemplary embodiment of the absorbent article in a fastened and unfastened position respectively. Here, the fastener 36 is shown attached to the extension 15. In the fastened position, the extension 15 folds together as the back 14 and front 12 sections are drawn together, and the fastener attaches to the extension 15 or the front stretch panel 28 to secure the adjusted waist circumference in the fastened position.

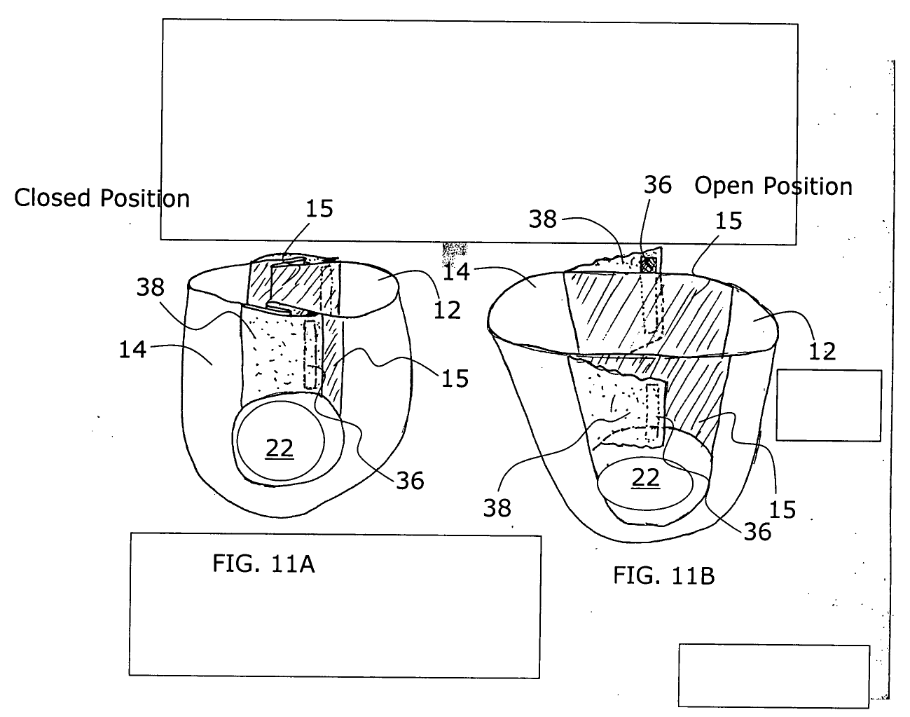

FIGS. 11A and 11B show another exemplary embodiment of the absorbent article in a fastened and unfastened position respectively. Here, a flap 38 is formed from the back section 14, and a fastener 36 is on the interior facing side of the flap. Again, the extension 15 folds together as the front and back sections are drawn together to adjust the waist of the article. The fastener on the flap secures the article in the adjusted position by fastening to the extension itself.

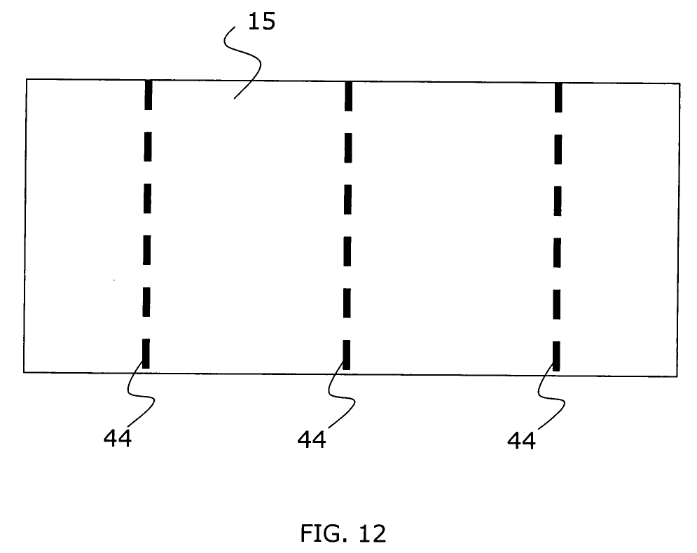

An exemplary embodiment of the extension 15 is shown in FIG. 12, where the extension contains tear lines 44. These tear lines function to assist in tearing the extension to facilitate removal of soiled articles. These tear lines may be perforations across the width of the extension. To better maintain structural integrity, the perforations at the edges of the extension may be spaced further apart, while the perforations in the center may be closer spaced so that it is more difficult to initiate tearing along a tear line than it is to continue tearing through the center.

The figures include the following reference numbers, provided with a summarizing label for convenience.
8 Interior side
10 Exterior side
12 front section
14 back section
15 extension
16 front portion of extension
17 back portion of extension
18 core
20 leg gathers
22 leg holes
24 bond between front portion of extension and front stretch panel
25 bond between extension and front stretch panel
26 bond between back portion of extension and back stretch panel
27 bond between extension and back stretch panel
28 front stretch panel
30 back stretch panel
32 front waistband
34 back waistband
36 fastener
38 flap
42 extended front stretch panel
44 tear line
46 bond between back stretch panel, extended front stretch panel and back section
48 bond between extended front stretch panel and front section
50 complementary fastener component

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An absorbent article comprising:
a front section and a back section, the front and back sections each configured to be positioned against the front and back, respectively, of a wearer of the absorbent article;
at least one extension extending between the front and back sections, the extension having ends coupled to the front and back sections, wherein the front and back sections and the extension together define a continuous waist of the absorbent article; and
a fastener applied to the article, wherein a portion of said waist folds together to adjust the size of the waist, the fastener being releasably attachable to the waist, thereby securing the adjusted size of the waist.

2. The absorbent article of claim 1 wherein the fastener is applied to the front section, the back section, or the extension of the article.

3. The absorbent article of claim 1 wherein the fastener is applied to a flap, wherein the flap is defined by a continuation of the back section of the article.

4. The absorbent article of claim 1 wherein the fastener is applied to the extension.

5. The absorbent article of claim 1 wherein the fastener is applied to the back section of the article.

6. The absorbent article of claim 1 wherein the fastener is a component of a mechanical closure system.

7. The absorbent article of claim 1 wherein the fastener is an adhesive.

8. The absorbent article of claim 1 wherein the extension comprises an elastic material.

9. The absorbent article of claim 1 wherein the extension comprises a non-elastic material.

10. The absorbent article of claim 1 wherein the extension comprises an adjustable panel component and a stretch panel component.

11. The absorbent article of claim 1, comprising a pair of extensions, wherein the waist is defined by said extensions on both sides of the wearer between the front and back sections.

12. The absorbent article of claim 1 comprising fasteners on each of said extensions.

13. The absorbent article of claim 1 wherein the extension extends, in an unfastened configuration, to create a waist circumference larger than an adjusted size of the waist.

14. The absorbent article of claim 13 wherein the waist circumference in the unfastened configuration allows the wearer to position and remove the absorbent article without disrupting the continuous waist.

15. The absorbent article of claim 1 wherein the extension comprises at least one tear line extending across the extension to facilitate a break in said waist.

16. The absorbent article of claim 15 wherein the tear line comprises perforations.

17. The absorbent article of claim 1 wherein the front and back sections comprise leg gathers for surrounding the leg of the wearer.

18. The absorbent article of claim 1 wherein an absorbent core extends along a portion of the front and back sections.

19. The absorbent article of claim 1 comprising plural extensions, wherein the extensions are formed from a non-woven material.

20. A method of making an absorbent article comprising:
forming front and back sections of an absorbent article each configured to be positioned against the front and back, respectively, of a wearer of the absorbent article;
coupling the ends of an extension to the front and back sections, creating a continuous waist of the absorbent article defined by the front and back sections and the extension, the extensions allowing a portion of the waist to fold together to adjust the size of the waist; and
attaching a fastener to the article in a location such that the fastener functions to secure the waist in the adjusted size.

21. The method of claim 20 further comprising forming a flap from the back section by attaching one end of the extension to the back section in a bond region of the back section, said flap formed of a portion of the back section between the bond region and the end of the back section.

22. The method of claim 20 wherein the step of coupling the extension comprises bonding the ends of the extension to the front and back sections of the absorbent article.

23. The method of claim 20 wherein step of attaching a fastener to the article comprises securing a fastener to a flap and bonding the flap to the waist of the absorbent article.

24. The method of claim 20 further comprising forming a tear-line across the extension.
